(19) United States
(12) Patent Application Publication
(10) Pub. No.: US 2022/0233656 A1
(43) Pub. Date: Jul. 28, 2022

(54) ANTIBACTERIAL COMPOSITION

(71) Applicants: NATIONAL UNIVERSITY CORPORATION TOKYO MEDICAL AND DENTAL UNIVERSITY, Bunkyo-ku, Tokyo (JP); WAKO FILTER TECHNOLOGY CO., LTD., Chiyoda-ku, Tokyo (JP)

(72) Inventors: Yasunari MIYAZAKI, Bunkyo-ku, Tokyo (JP); Hiroki KAGESHIMA, Nagareyama-shi, Chiba (JP); Akira SAITO, Chiyoda-ku, Tokyo (JP)

(73) Assignees: NATIONAL UNIVERSITY CORPORATION TOKYO MEDICAL AND DENTAL UNIVERSITY, Bunkyo-ku, Tokyo (JP); WAKO FILTER TECHNOLOGY CO., LTD., Chiyoda-ku, Tokyo (JP)

(21) Appl. No.: 17/613,973

(22) PCT Filed: May 27, 2020
(86) PCT No.: PCT/JP2020/020901

§ 371 Date: Nov. 24, 2021

(30) Foreign Application Priority Data
May 27, 2019 (JP) 2019-098528

(51) Int. Cl. A61K 38/47; A61K 47/61; A61P 31/04

(52) U.S. Cl. CPC A61K 38/47; A61P 31/04; A61K 47/61

(57) ABSTRACT

The present invention relates to an antibacterial composition and the like having antibacterial property against Acinetobacter. The present invention provides an antibacterial composition comprising a complex of lysozyme and chitosan bound together, with antibacterial property against Acinetobacter; and a pharmaceutical composition for treating or preventing an Acinetobacter infection, comprising a complex of lysozyme and chitosan bound together.

### TECHNICAL FIELD

The present invention relates to an antibacterial composition and the like having antibacterial property against Acinetobacter.

### BACKGROUND ART

Nontuberculous mycobacteria such as Pseudomonas aeruginosa or MRSA are infectious bacteria that cause respiratory tract infections. These infections are considered to be a problem because they become intractable. It has recently been shown that a complex of lysozyme and chitosan bound together is effective against Pseudomonas aeruginosa and MRSA (Patent Literature 1). On the other hand, for Acinetobacter, which is recognized as one of the most important bacteria in severe pneumonia, although there are drugs such as colistin and polymyxin B that have been shown to be effective to some extent, they have not been approved in Japan due to nephrotoxic side effects and other reasons, and it has been considered difficult to treat Acinetobacter infections with common antibacterial drugs (Non Patent Literature 1). Furthermore, no effective drug has yet been found for Acinetobacter that has become resistant (multidrug-resistant Acinetobacter infection), and the invention of a novel drug has been awaited.

### CITATION LIST

### Patent Literatures

Patent Literature 1: International Publication No. 2017/138476 Pamphlet

Non Patent Literatures

Non Patent Literature 1: Multidrug-Resistant Acinetobacter Infection Q&A, Infectious Disease Surveillance Center Homepage, National Institute of Infectious Diseases [http://idsc.nih.go.jp/disease/MDRA/QA01.html]

### SUMMARY OF INVENTION

An object of the present invention is to provide an antibacterial composition against Acinetobacter, especially multidrug-resistant Acinetobacter. The present invention also provides an antibacterial composition that exhibits effective treating and preventing effects against Acinetobacter infections or multidrug-resistant Acinetobacter infections, a method of treatment and prevention thereof, and a method of using the antibacterial composition in the production of antibacterial agents.

The inventors of the present application overturned the common general technical knowledge that general antibacterial drugs as described above are not effective against Acinetobacter, and used a complex of lysozyme and chitosan, which are natural food additives with high safety, to examine its antibacterial property against Acinetobacter. Then, it was found that the complex showed an excellent antibacterial effect against Acinetobacter. Thus, the present invention has been completed.

More specifically, the present invention can be in the following aspects.
[1] An antibacterial composition including a complex of lysozyme and chitosan bound together, with antibacterial property against Acinetobacter.
[2] The antibacterial composition according to [1] described above, wherein the Acinetobacter is multidrug-resistant Acinetobacter.
[3] The antibacterial composition according to [1] or [2] described above, wherein a concentration of the complex in 1 mL of the antibacterial composition is 200 μg/mL or more.
[4] The antibacterial composition according to any one of [1] to [3] described above, wherein the antibacterial property has bacteria-killing property.
[5] Rinse water including the antibacterial composition according to any one of [1] to [4] described above.
[6] A pharmaceutical composition for treating or preventing an Acinetobacter infection, including a complex of lysozyme and chitosan bound together.
[7] The pharmaceutical composition according to [6] described above, wherein the Acinetobacter infection is a multidrug-resistant Acinetobacter infection.
[8] Use of a complex of lysozyme and chitosan bound together in production of a pharmaceutical composition for treating or preventing an Acinetobacter infection.

### Advantageous Effects of Invention

### Advantageous Effects of Invention

The present invention provides the effect of removing, suppressing, or killing Acinetobacter and the effect of suppressing its growth. In particular, the complex of lysozyme and chitosan bound together of the present invention has the effects of not only suppressing the growth of Acinetobacter, but also significantly killing it. Therefore, according to the present invention, Acinetobacter infections and multidrug-resistant Acinetobacter infections can be cured and/or prevented. In addition, lysozyme is widely used as a safe and natural food additive, and the antibacterial composition using a complex of this lysozyme and chitosan can reassure patients who use it and reduce their burden.

### BRIEF DESCRIPTION OF DRAWINGS

FIG. 1 is a schematic diagram of the reaction in synthesizing a complex of lysozyme and chitosan bound together.

FIG. 2A is a diagram showing the bacteria-killing effects of a lysozyme-chitosan complex (LYZOX (registered trademark)) and a mixture of lysozyme and chitosan on Pseudomonas aeruginosa (NBRC 13275) in standard medium.

FIG. 2B is a diagram showing the bacteria-killing effects of a lysozyme-chitosan complex (LYZOX (registered trademark)) and a mixture of lysozyme and chitosan on Pseudomonas aeruginosa (PAO1) in standard medium.

FIG. 2C is a diagram showing the bacteria-killing effects of a lysozyme-chitosan complex (LYZOX (registered trademark)) and a mixture of lysozyme and chitosan on Acinetobacter (JCM 6841) in standard medium.

FIG. 2D is a diagram showing the bacteria-killing effect of the lysozyme-chitosan complex (LYZOX (registered trademark)) on Pseudomonas aeruginosa (NBRC 13275) at various concentrations.

FIG. 3A is a diagram showing the growth suppression effect on Pseudomonas aeruginosa (NBRC 13275) in standard medium by the lysozyme-chitosan complex (LYZOX (registered trademark)), lysozyme alone, chitosan alone, and a mixture of lysozyme and chitosan.

FIG. 3B is a diagram showing the growth suppression effect on Pseudomonas aeruginosa (PAO1) in standard medium by the lysozyme-chitosan complex (LYZOX (registered trademark)), lysozyme alone, chitosan alone, and a mixture of lysozyme and chitosan.

FIG. 3C is a diagram showing the growth suppression effect on Acinetobacter (JCM 6841) in standard medium by the lysozyme-chitosan complex (LYZOX (registered trademark)), lysozyme alone, chitosan alone, and a mixture of lysozyme and chitosan.

FIG. 3D is a diagram showing the growth suppression effect of the lysozyme-chitosan complex (LYZOX (registered trademark)) on Pseudomonas aeruginosa (NBRC 13275) at various concentrations.

FIG. 4A is a diagram showing the results of measuring the integrity of the cell membrane of Pseudomonas aeruginosa (NBRC 13275) in contact with the lysozyme-chitosan complex (LYZOX (registered trademark)) at various concentrations, in terms of absorbance.

FIG. 4B is a diagram showing the results of measuring the integrity of the cell membrane of Acinetobacter (JCM 6841) in contact with the lysozyme-chitosan complex (LYZOX (registered trademark)) at various concentrations, in terms of absorbance.

FIG. 5A is a diagram showing the results of measuring the cell membrane damage of Pseudomonas aeruginosa (NBRC 13275) in contact with the lysozyme-chitosan complex (LYZOX (registered trademark)) at various concentrations by NPN assay (extracellular membrane permeability test).

FIG. 5B is a diagram showing the results of measuring the cell membrane damage of Acinetobacter (JCM 6841) in contact with the lysozyme-chitosan complex (LYZOX (registered trademark)) at various concentrations by NPN assay (extracellular membrane permeability test).

FIG. 6A is a diagram showing the results of measuring the cell membrane damage of Pseudomonas aeruginosa (NBRC 13275) in contact with the lysozyme-chitosan complex (LYZOX (registered trademark)) at various concentrations by ONPG assay (intracellular membrane permeability test).

FIG. 6B is a diagram showing the results of measuring the cell membrane damage of Acinetobacter (JCM 6841) in contact with the lysozyme-chitosan complex (LYZOX (registered trademark)) at various concentrations by ONPG assay (intracellular membrane permeability test).

FIG. 7A provides images showing the results of measuring the cell viability of Pseudomonas aeruginosa (NBRC 13275) in contact with the lysozyme-chitosan complex (LYZOX (registered trademark)) at various concentrations, visualized with a confocal laser scanning microscope (CLSM).

FIG. 7B provides images showing the results of measuring the cell viability of Acinetobacter (JCM 6841) in contact with the lysozyme-chitosan complex (LYZOX (registered trademark)) at various concentrations, visualized with a confocal laser scanning microscope (CLSM).

FIG. 8A provides images showing the morphological changes of Pseudomonas aeruginosa (NBRC 13275) obtained with a scanning electron microscope when the lysozyme-chitosan complex (LYZOX (registered trademark)), lysozyme alone, chitosan alone, and a mixture of lysozyme and chitosan were added, respectively.

FIG. 8B provides images showing the morphological changes of Acinetobacter (JCM 6841) obtained with a scanning electron microscope when the lysozyme-chitosan complex (LYZOX (registered trademark)), lysozyme alone, chitosan alone, and a mixture of lysozyme and chitosan were added, respectively.

FIG. 9A provides images showing the morphological changes of Pseudomonas aeruginosa (NBRC 13275) obtained with a transmission electron microscope when the lysozyme-chitosan complex (LYZOX (registered trademark)), lysozyme alone, chitosan alone, and a mixture of lysozyme and chitosan were added, respectively.

FIG. 9B provides images showing the morphological changes of Acinetobacter (JCM 6841) obtained with a transmission electron microscope when the lysozyme-chitosan complex (LYZOX (registered trademark)), lysozyme alone, chitosan alone, and a mixture of lysozyme and chitosan were added, respectively.

FIG. 10A is a diagram showing the effects of the drug resistance acquisition test on Pseudomonas aeruginosa (NBRC 13275) in standard medium, using the lysozyme-chitosan complex (LYZOX (registered trademark)) and a mixture of lysozyme and chitosan.

FIG. 10B is a diagram showing the effects of the drug resistance acquisition test on Pseudomonas aeruginosa (PAO1) in standard medium, using the lysozyme-chitosan complex (LYZOX (registered trademark)) and a mixture of lysozyme and chitosan.

FIG. 10C is a diagram showing the effects of the drug resistance acquisition test on Acinetobacter (JCM 6841) in standard medium, using the lysozyme-chitosan complex (LYZOX (registered trademark)) and a mixture of lysozyme and chitosan.

### DESCRIPTION OF EMBODIMENTS

An embodiment of the present invention is described in detail below.

### Antibacterial Composition

The present invention relates to an antibacterial composition including a complex of lysozyme and chitosan bound together, with antibacterial property against Acinetobacter. Hereinafter, the present invention is specifically described.

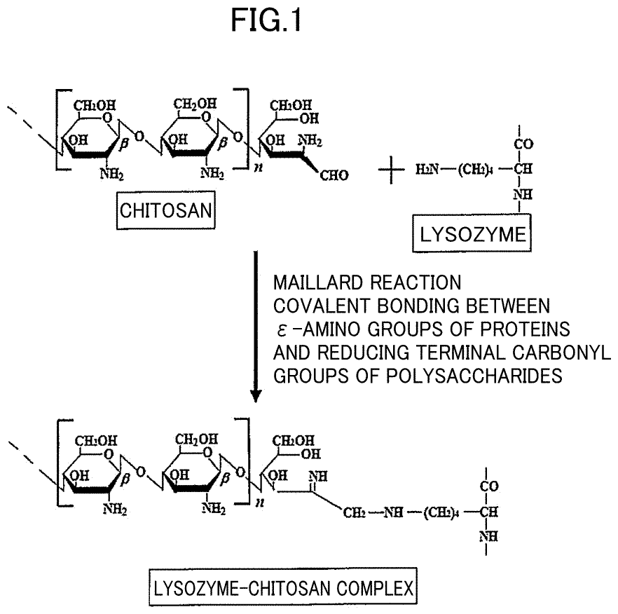

The “complex of lysozyme and chitosan bound together” is a complex in which lysozyme and chitosan are bound by the Maillard reaction or the like, for example (see FIG. 1). By bonding lysozyme and water-soluble chitosan through the Maillard reaction, most or all of the antigenic structure in lysozyme is masked, and thus the lysozyme-chitosan complex is less likely to cause allergies even when ingested by humans. Otherwise, the above complex can be obtained by covalently bonding lysozyme and chitosan using a cross-linking agent.

Here, “lysozyme” is an enzyme that hydrolyzes mucopolysaccharides, and it is possible to preferably use lysozyme derived from chickens and lysozyme derived from humans.

The upper limit of the molecular weight of lysozyme is, for example, 30,000 Da or less, and more preferably it may be 25,000 Da or less, 20,000 Da or less, 18,000 Da or less, or 15,000 Da or less. In addition, the lower limit of the molecular weight of lysozyme does not have to be restricted, but may be, for example, 1,000 Da or more, and preferably it may be 5,000 Da or more, 10,000 Da or more, or 12,000 Da or more. The range of the molecular weight of the lysozyme can be between any of the above upper limit values and lower limit values, but can be, for example, 1000 Da to 30,000 Da, preferably 5,000 Da to 20,000 Da, and more preferably 10,000 Da to 15,000 Da.

“Chitosan” is poly-β1→4-glucosamine represented by the following chemical formula (I) ((C₆H₁₁NO₄)ₙ, CAS registration number 9012-76-4).

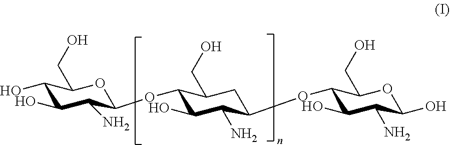

This chitosan is water soluble. The upper limit of the molecular weight of chitosan is, for example, 30,000 Da or less, and more preferably it may be 20,000 Da or less, 15,000 Da or less, 10,000 Da or less, or 7,000 Da or less. In addition, the lower limit of the molecular weight of chitosan does not have to be restricted, but may be, for example, 300 Da or more, and preferably it may be 500 Da or more, 1,000 Da or more, or 3,000 Da or more. The range of the molecular weight of the chitosan can be between any of the above upper limit values and lower limit values, but can be, for example, 300 Da to 30,000 Da, preferably 500 Da to 15,000 Da, more preferably 1,000 Da to 10,000 Da, and particularly preferably 3,000 Da to 7,000 Da. In terms of antibacterial property, chitosan with higher molecular weight is more advantageous, while in terms of ease of production, chitosan with lower molecular weight has better solubility and stability, so that chitosan with lower molecular weight is more advantageous.

Note that the term “chitosan” here includes chitosan oligosaccharides and glucosamine as well as the above chitosan. Chitosan oligosaccharides are a series of several D-glucosamines as shown in the above formula (I), and mean low-molecular-weight chitosan or chitosan in the narrow sense that has been hydrolyzed with hydrochloric acid or enzymes.

The above cross-linking agent includes, for example, amine-reactive cross-linking agents (such as alkoxyamines), carbonyl-reactive cross-linking agents (such as hydrazine compounds), and sulfhydryl-reactive cross-linking agents.

The mass ratio of lysozyme/chitosan is suitable, for example, to be 99/1 to 1/99, preferably 90/10 to 10/90, more preferably 80/20 to 20/80, further preferably 60/40 to 40/60, and particularly preferably 50/50.

The specific method of producing a complex of lysozyme and chitosan bound together is as follows, for example. First, lysozyme and chitosan in the above mass ratio are mixed and dissolved in water, and the total mass of lysozyme and chitosan in the resulting aqueous solution is prepared to be 5 to 30% by mass. The resulting aqueous solution is lyophilized to make a powder. The powder obtained can be subjected to the Maillard reaction, for example, for 2 to 20 days, more preferably for 7 to 14 days, under the conditions of a temperature of, for example, 50 to 80° C., preferably 55 to 65° C., and a relative humidity of, for example, 50 to 80%, preferably 60 to 70%, to produce the complex of lysozyme and chitosan bound together of the present invention.

Whether or not the lysozyme-chitosan complex of the present embodiment is produced can be confirmed by various known methods, and for example, plates obtained by SDS (Sodium dodecyl sulfate) or SDS-PAGE (Sodium dodecyl sulfate-polyacrylamide) polyacrylamide electrophoresis can be stained and treated to confirm the production of a macromolecular substance that is a protein-chitosan complex.

“Acinetobacter” is an aerobic, Gram-negative bacterium in the genus Acinetobacter, a family of Gram-negative bacillus bacteria. Acinetobacter in the genus Acinetobacter is known to include Acinetobacter calcoaceticus, Acinetobacter lwoffii, Acinetobacter baumannii, and the like, with Acinetobacter baumannii (JCM 6841) being particularly known as a causative agent of infections, especially respiratory infections. The present invention has a characteristic that it is particularly effective against Acinetobacter spp. and Acinetobacter, as well as against multidrug-resistant Acinetobacter, which does not acquire drug resistance.

The present invention has antibacterial property against “Acinetobacter” and “multidrug-resistant Acinetobacter.” Here, the antibacterial property broadly includes removing bacteria, killing bacteria, suppressing bacteria, and the like, and mean all conditions in which bacteria are not allowed to grow, at least when the bacteria count is equal to or less than that. Note that removing bacteria means the removal of bacteria in general, and its meaning includes killing bacteria. Killing bacteria means killing at least some of the bacteria. Therefore, in the present invention, the antibacterial composition means including bacteria-removing compositions, bacteria-killing compositions, bacteria-removing agents, bacteria-killing agents, and antibacterial agents.

The present invention is further effective in treating and preventing “Acinetobacter infection” and “multidrug-resistant Acinetobacter infection.” Here, “treating” includes complete cure of inflammation, which is a target of the present invention, as well as suppression of inflammation and reduction of the severity thereof “Preventing” includes precluding the recurrence of inflammation being a target of the present invention after it has been cured, as well as when there is no history of the inflammation, which is a target of the present invention.

The antibacterial composition of the present invention can optionally contain one or more additional active ingredients in addition to the above complex. The additional active ingredients include those that act as active ingredients by themselves, as well as those that do not act as active ingredients by themselves but exert their effects (auxiliary agents) when used in combination with the above complex, which is an active ingredient of the present invention. The additional active ingredients include, for example, terpene alcohols, fatty acids, and/or salts of the fatty acids. The addition of these terpene alcohols, fatty acids, and/or salts of the fatty acids, when combined with the complex of the present invention, can produce additive effects and synergistic effects. As terpene alcohols, for example, terpinen-4-ol, hinokitiol, geraniol, menthol, and the like can be mentioned. As fatty acids, for example, fatty acids having 8 to 12 carbon atoms, preferably decanoic acid (capric acid) or lauric acid, can be mentioned. As salts of fatty acids, for example, sodium salts, potassium salts, magnesium salts, calcium salts, and the like can be mentioned. In addition, essential oils obtained from lemongrass, spearmint, geranium, shiso, and the like, which are known to be effective against inflammation caused by neutrophils, and glucosamine may be added as the additional active ingredients.

The antibacterial composition of the present invention may optionally contain one or more additional components in addition to the above complex. As the additional components, for example, excipients, binders, emulsifiers, solvents, adhesives, disintegrants, thickeners, lubricants, colorants, flowable agents, humectants, and other additives may be added.

The humectants include, for example, glycerin, butylene glycol, collagen, hyaluronic acid, and ceramide.

The binders include, for example, cellulose, methyl cellulose, hydroxyethyl cellulose, and sodium carboxymethyl cellulose.

The emulsifiers include, for example, lecithin, polyethylene glycol (PG), PG-hydrogenated castor oil, glycerin fatty acid esters, sorbitan fatty acid esters, ceteth, and the like.

The solvents include, for example, water, ethanol, 1-butanol, 2-butanol, 1-propanol, 2-propanol, 1-pentanol, and the like.

The lysozyme-chitosan complex of the present invention is suitable to be contained in 1 mL of the antibacterial composition of the present invention, for example, at 10 μg/mL to 200,000 μg/mL, preferably 100 μg/mL to 100,000 μg/mL, more preferably 50 μg/mL to 50,000 μg/mL, further preferably 100 μg/mL to 10,000 μg/mL, and particularly preferably 200 μg/mL to 5,000 μg/mL. In addition, the additional active ingredients are suitable to be contained in the antibacterial composition of the invention, for example, at 0.001% by mass to 5.0% by mass, preferably 0.002% by mass to 0.2% by mass, more preferably 0.005% by mass to 0.1% by mass, further preferably 0.01% by mass to 0.05% by mass, and particularly preferably 0.02% by mass to 0.04% by mass. Furthermore, the appropriate content of the additional components needs to be appropriately adjusted depending on each component, and is suitable to be contained, for example, at 0.1% by mass to 90% by mass, preferably 1% by mass to 70% by mass, and more preferably 10% by mass to 50% by mass. Note that most of the antibacterial composition of the present invention can be a solvent such as water, and the solvent is suitable to be contained in the antibacterial composition of the present invention, for example, at 95% by mass or more, preferably 98% by mass or more, more preferably 99% by mass or more, and further preferably 99.5% by mass or more. In addition, fatty acids having 8 to 12 carbon atoms, such as lauric acid and decanoic acid (capric acid), or salts of the fatty acids, are suitable to be contained in the antibacterial composition of the present invention, for example, at 0.001% by mass to 5% by mass, preferably 0.005% by mass to 1% by mass, more preferably 0.01% by mass to 0.5% by mass, and particularly preferably 0.04% by mass to 0.2% by mass.

### Treatment/Prevention Method, and Pharmaceutical Composition Therefore

The present invention can include a method of treating, suppressing, or preventing Acinetobacter infections, preferably multidrug-resistant Acinetobacter infections, by administering to a subject a pharmaceutical composition containing a complex of lysozyme and chitosan bound together, and a pharmaceutical composition therefor. The treatment, suppression, or prevention effect is attributed to the above-mentioned antibacterial property of the complex of lysozyme and chitosan bound together, and is considered to be particularly effective for respiratory diseases. Here, the pharmaceutical composition includes the above-mentioned antibacterial composition, and is all the same as the above-mentioned antibacterial composition in that it may contain additional active ingredient and the like, as well as the content, the content ratio, and the like.

Here, the “subject” includes not only humans but also mammals such as cats, dogs, monkeys, cows, and horses.

Each definition of lysozyme, chitosan, and the like, the additional active ingredients and additional components, dose, dosage form, administration method, and the like are as described above.

### Dose, Dosage Form, and Administration Method

For the antibacterial composition and pharmaceutical composition of the present invention, although it varies depending on their dosage form, target of administration, route of administration, target disease, symptoms, and the like, the daily dose is, for example, 0.1 to 20 mg/kg body weight, preferably 0.2 to 10 mg/kg body weight, and further preferably 0.5 to 10 mg/kg body weight, and this amount is desirably administered once to several times a day (for example, two, three, four, or eight times a day), in the case of the form of a spray for spraying on human skin, for example. This dose can be preferably applied not only to sprays but also to other formulations such as creams as described below.

The antibacterial composition and pharmaceutical composition of the present invention can be administered orally or parenterally, and no special techniques are required for their formulation, and they can be formulated using general-purpose techniques. The dosage forms administered include creams, ointments, cataplasms, liquids, sprays, gels, injections, tablets, suppositories, capsules, granules, powders, ophthalmic solutions, eye ointments, and the like, and creams, ointments, cataplasms, liquids, and sprays are particularly preferable.

For the antibacterial composition and pharmaceutical composition of the present invention, the administration method can be appropriately selected according to the above-mentioned dosage form. The administration method may be a known administration method, and for example, in the case of a cream, the pharmaceutical composition of the present invention may be applied to an inflamed site at the above dose.

### Use

The present invention may also include the use of a complex of lysozyme and chitosan bound together in the production of a pharmaceutical composition for treating or preventing multidrug-resistant Acinetobacter infection.

In the production of a pharmaceutical composition for treating or preventing Acinetobacter infections, preferably multidrug-resistant Acinetobacter infections, the complex of lysozyme and chitosan bound together is mixed with the components of the pharmaceutical composition other than the complex to form the pharmaceutical composition. For the mixing method, any known method can be used, and for example, in the case of a liquid, the complex of lysozyme and chitosan bound together and any of the above optional additional components are added and mixed in a solvent such as water, and if necessary, an emulsifier is added and mixed to form a dispersant or emulsion, thereby preparing a liquid.

This antibacterial composition can be used as rinse water for nebulizers. This allows the antibacterial composition to be sprayed directly onto the infected area, thereby suppressing the growth of infectious bacteria.

In addition, the antibacterial composition and pharmaceutical composition of the present embodiment have, for example, antibacterial effects and bacterial growth suppression effects against Acinetobacter, preferably multidrug-resistant Acinetobacter, and treatment, suppression, and prevention effects against infections caused by these, as well as preclusion effects against the acquisition of drug resistance.

Hereinafter, the present invention is described in more detail with reference to Examples, but the present invention is not limited to these Examples. Note that in the present specification and drawings, the complex of lysozyme and chitosan bound together is also referred to as lysozyme-chitosan complex, or LYZOX (registered trademark of Wako Filter Technology Co., Ltd.).

### EXAMPLES

### Preparation of Samples

### Lysozyme-Chitosan Complex (LYZOX (Registered Trademark)) Solution

Commercially available LYZOX (registered trademark, manufactured by Wako Filter Technology Co., Ltd.) was used as a solution of a complex of lysozyme and chitosan bound together (lysozyme-chitosan complex). Commercially available LYZOX (registered trademark) is a powder containing chicken-derived lysozyme (about 14,000 Da) and about 5,000 Da of water-soluble chitosan (chitosan oligosaccharide) in a mass ratio of 1:1. Specifically, the above lysozyme and water-soluble chitosan were mixed and dissolved in water, then lyophilized to form a powder, and further subjected to the Maillard reaction under the conditions of temperature, humidity, and days sufficient for the Maillard reaction to be completely finished, thereby obtaining the lysozyme-chitosan complex (LYZOX (registered trademark)) used in the present invention. The lysozyme-chitosan complex (LYZOX (registered trademark)) was mixed with a predetermined solvent to achieve the desired concentration of the lysozyme-chitosan complex in each Experimental Example, thereby obtaining the target lysozyme-chitosan complex solution (LYZOX (registered trademark)).

### Solution of Lysozyme Alone

For the solution of lysozyme alone, lysozyme (manufactured by Nippon Biocon Ltd., Aichi Prefecture) was mixed with a predetermined solvent to achieve the desired concentration of the lysozyme in each Experimental Example, thereby obtaining the target solution of lysozyme alone.

### Solution of Chitosan Alone

For the solution of chitosan alone, chitosan oligosaccharide (KIMICA Chitosan Oligosaccharide COS-A manufactured by KIMICA Corporation) was used as chitosan, and in each Experimental Example, the chitosan oligosaccharide was mixed with a predetermined solvent to achieve the desired concentration, thereby obtaining the target solution of chitosan alone.

### Solution of a Mixture of Lysozyme and Chitosan

The mixture of lysozyme and chitosan was prepared as follows. Both were mixed so that the mass ratio of lysozyme alone:chitosan alone was 1:1, and a predetermined solvent was further mixed so that the mixture, lysozyme, and chitosan had a desired concentration.

### Acinetobacter Bacterial Suspension

Unless otherwise noted, Acinetobacter baumannii (JCM 6841) was grown in trypsin soy broth (TSB) overnight at 37° C. and centrifuged during the logarithmic growth phase to collect the bacteria. The obtained bacteria were mixed with a predetermined amount of each solvent in each test to obtain an Acinetobacter bacterial suspension.

Pseudomonas aeruginosa Bacterial Suspension

Unless otherwise noted, a bacterial suspension was prepared in the same manner as the Acinetobacter bacterial suspension described above, except that Pseudomonas aeruginosa (NBRC 13275 or PAO1) was used instead of Acinetobacter baumannii (JCM 6841). The details of each specific Experimental Example are as shown below.

### Absorbance Measurement

Unless otherwise noted, the spectrophotometer ASV11D manufactured by AS ONE Corporation was used for the measurement of the absorbance in the present Examples. In the actual measurement of absorbance, the absorbance was calculated by diluting the sample as necessary in consideration of the absorbance measurable with the spectrophotometer.

### Experimental Example 1 (Bacteria-Killing Test)

The bacteria-killing property of the lysozyme-chitosan complex (LYZOX (registered trademark)) against Acinetobacter and the like was measured in comparison with a mixture of lysozyme and chitosan and the like.

Specifically, to the above-mentioned lysozyme-chitosan complex (LYZOX (registered trademark)) solution (2,000 μg/mL, physiological saline was used as a solvent) and a solution of a mixture of lysozyme and chitosan (solution of lysozyme alone [1,000 μg/mL] and solution of chitosan alone [1,000 μg/mL], physiological saline was used as a solvent), 30 μL of 1.0 M phosphate buffered saline (pH 7.2) was added per 1.0 mL of the solution to adjust to a neutral pH (7.0 to 7.3), thereby preparing each solution. As a control, physiological saline (0.9% NaCl solution) was used. The Acinetobacter bacterial suspension or Pseudomonas aeruginosa bacterial suspension was prepared as follows. Pseudomonas aeruginosa (NBRC 13275 or PAO1) or Acinetobacter baumannii (JCM 6841) was grown overnight at 37° C. in trypsin soy broth (TSB), centrifuged during the logarithmic growth phase, and collected, and the bacteria were resuspended in physiological saline (0.9% NaCl), with such a final suspension concentration that the absorbance was 1.0 (10⁸ to 10⁹ CFU/mL) at 600 nm.

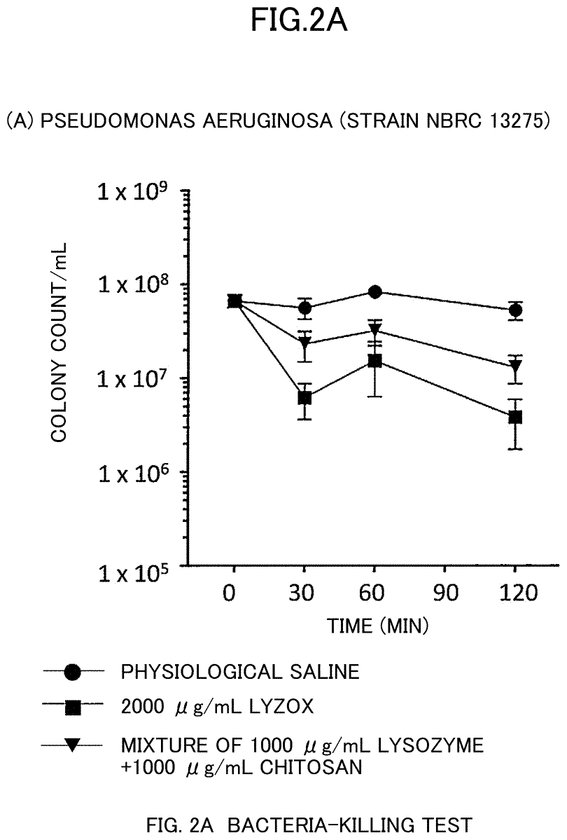

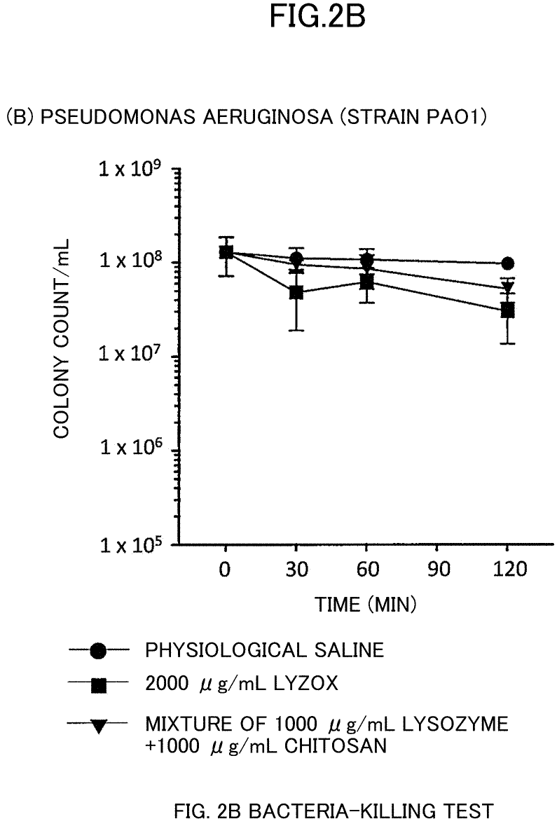

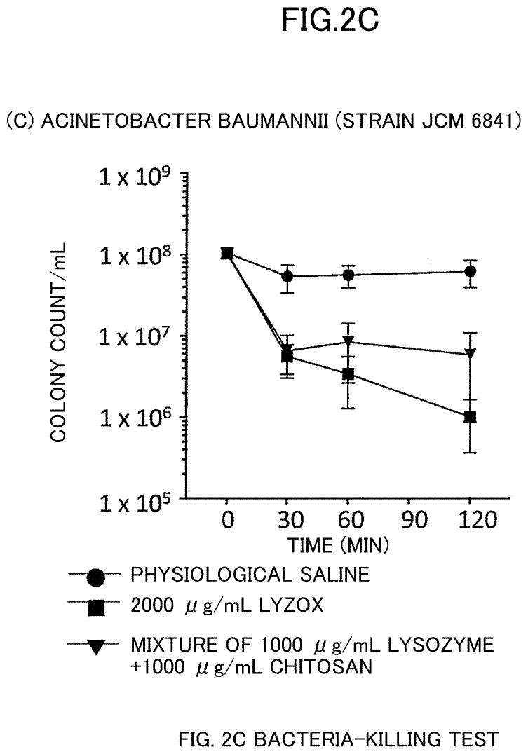

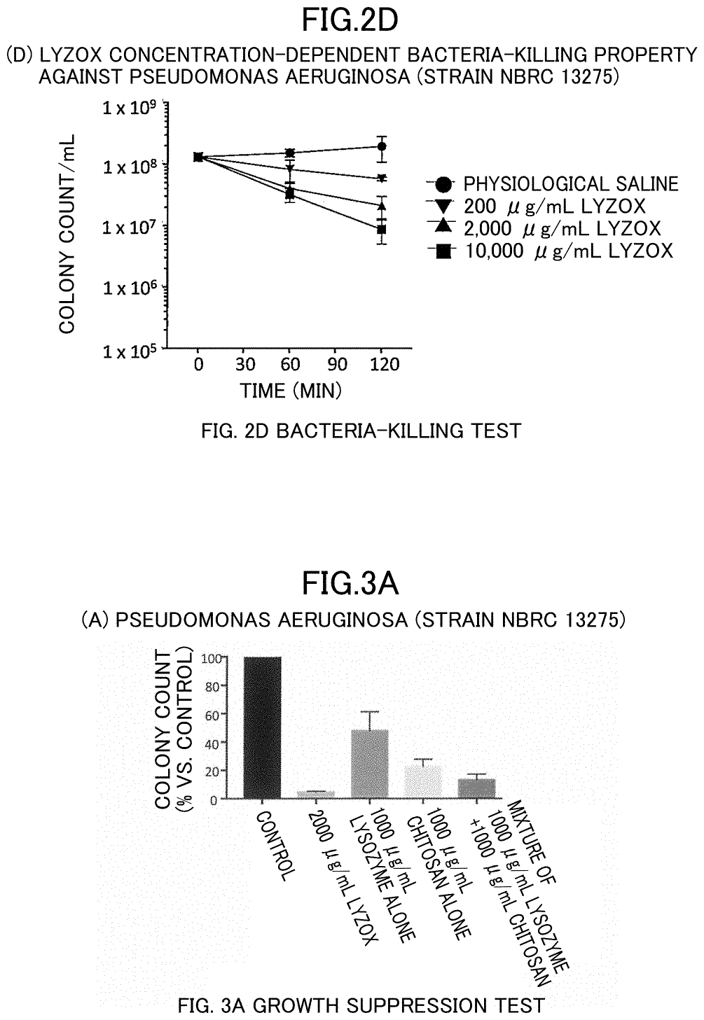

To 10 mL of each solution of lysozyme-chitosan complex or the like above, 0.1 mL of the above Acinetobacter bacterial suspension or Pseudomonas aeruginosa bacterial suspension (1×10⁷ to 10⁸ CFU) was added, and incubated at 37° C. using a water bath. At 0 minutes, 30 minutes, 60 minutes, and 120 minutes after the start of incubation, 0.1 mL of each solution was added to a solid medium (Mueller-Hinton medium) for overnight growth, and then the bacteria count was measured. The test of the bacterial suspensions against each solution was performed three times and measured. The results are shown in FIGS. 2A to 2C.

The results showed that in Acinetobacter (FIG. 2C), the lysozyme-chitosan complex significantly reduced the bacteria count than the control at 60 minutes and 120 minutes.

### Experimental Example 2 (Bacteria-Killing Test)

The bacteria-killing effect of Pseudomonas aeruginosa (NBRC 13275) on the lysozyme-chitosan complex (LYZOX (registered trademark)) at various concentrations was determined. Specifically, solutions with different concentrations (200 μg/mL, 2,000 μg/mL, and 10,000 μg/mL) were prepared as the above-described lysozyme-chitosan complex (LYZOX (registered trademark)) solution, using physiological saline (0.9% NaCl solution) as the solvent, and the experiment was repeated in the same way as in Experimental Example 1 above to measure the bacteria count of Pseudomonas aeruginosa (NBRC 13275). The results are shown in FIG. 2D. As shown in FIG. 2D, the lysozyme-chitosan complex showed concentration-dependent bacteria-killing activity.

### Experimental Example 3 (Growth Suppression Effect Test)

The growth suppression effect of the lysozyme-chitosan complex (LYZOX (registered trademark)) on Acinetobacter and the like was measured in comparison with lysozyme alone, chitosan alone, and a mixture of lysozyme and chitosan.

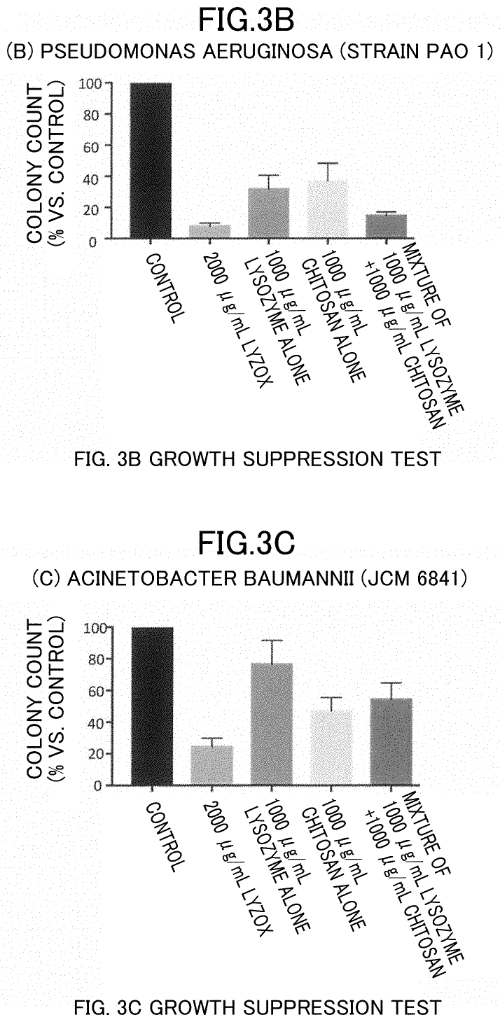

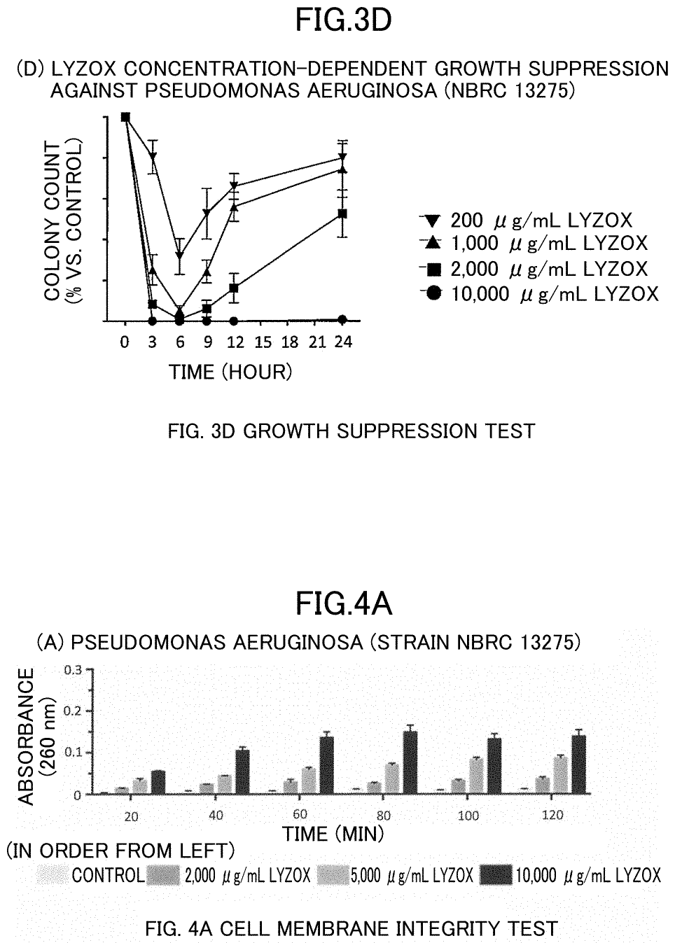

Specifically, the above-mentioned lysozyme-chitosan complex (LYZOX (registered trademark)) solution (2,000 μg/mL), a solution of lysozyme alone (1,000 μg/mL), a solution of chitosan alone (1,000 μg/mL), and a solution of a mixture of lysozyme and chitosan (solution of lysozyme alone [1,000 μg/mL] and solution of chitosan alone [1,000 μg/mL]) were prepared using a trypsin soy broth (TSB) solution (Japan Becton, Dickinson and Company) as a solvent, and 10 μL of 1.0 M phosphate buffered saline (pH 7.2) was added to each solution per 1.0 mL of the solution to adjust to a neutral pH (6.8 to 7.3), thereby preparing each solution. As a control, TSB medium without additives was used. The Acinetobacter bacterial suspension or Pseudomonas aeruginosa bacterial suspension was prepared in the same way as in Experimental Example 1 described above. To 10 mL of each solution of lysozyme-chitosan complex or the like above, 50 μL (2.5×10³ to 10⁴ CFU) of the above Acinetobacter bacterial suspension or Pseudomonas aeruginosa bacterial suspension diluted 2000-fold was further added, and incubated at 37° C. for 6 hours. After 6 hours, 0.1 mL of each solution was added to a solid medium (Mueller-Hinton medium) and allowed to grow overnight, and then the bacteria count was measured. The bacterial suspensions were tested on each solution 18 times for Pseudomonas aeruginosa (NBRC 13275) and 8 times each for Pseudomonas aeruginosa (PAO1) and Acinetobacter by duplicate (mean of triplicate measurements and SEM (standard error of sample mean)) in independent experiments. The results are shown in FIGS. 3A to 3C. Note that in order to match the mass concentration of each constituent component (lysozyme and chitosan), the antibacterial action was compared between lysozyme alone and chitosan alone, and the lysozyme-chitosan complex with twice the concentration of the lysozyme alone and chitosan alone.

The results showed that in Acinetobacter (FIG. 3C), the lysozyme-chitosan complex significantly reduced the bacteria count than the control.

### Experimental Example 4 (Growth Suppression Effect Test)

The growth suppression effect test of Pseudomonas aeruginosa (NBRC 13275) on the lysozyme-chitosan complex (LYZOX (registered trademark)) at various concentrations was measured. Specifically, solutions with different concentrations (2,000 μg/mL, 5,000 μg/mL, and 10,000 μg/mL) were prepared as the above-described lysozyme-chitosan complex (LYZOX (registered trademark)) solution, using TSB as the solvent, and the experiment was repeated in the same way as in Experimental Example 3 above to measure the bacteria count of Pseudomonas aeruginosa (NBRC 13275). The results are shown in FIG. 3D. As shown in FIG. 3D, the lysozyme-chitosan complex showed concentration-dependent growth suppression effect.

### Experimental Example 5 (Cell Membrane Integrity Test)

The measurement of absorbance at 260 nm (A₂₆₀ₙₘ) was used to estimate the amount of nucleic acid released from the cytoplasm, thereby evaluating the integrity of the cell membrane. When a bacterial cell membrane is compromised by an antibacterial agent, intracellular components are released into the extracellular space. The measurement of absorbance at 260 nm is used to estimate the amount of DNA and RNA released from the cytoplasm.

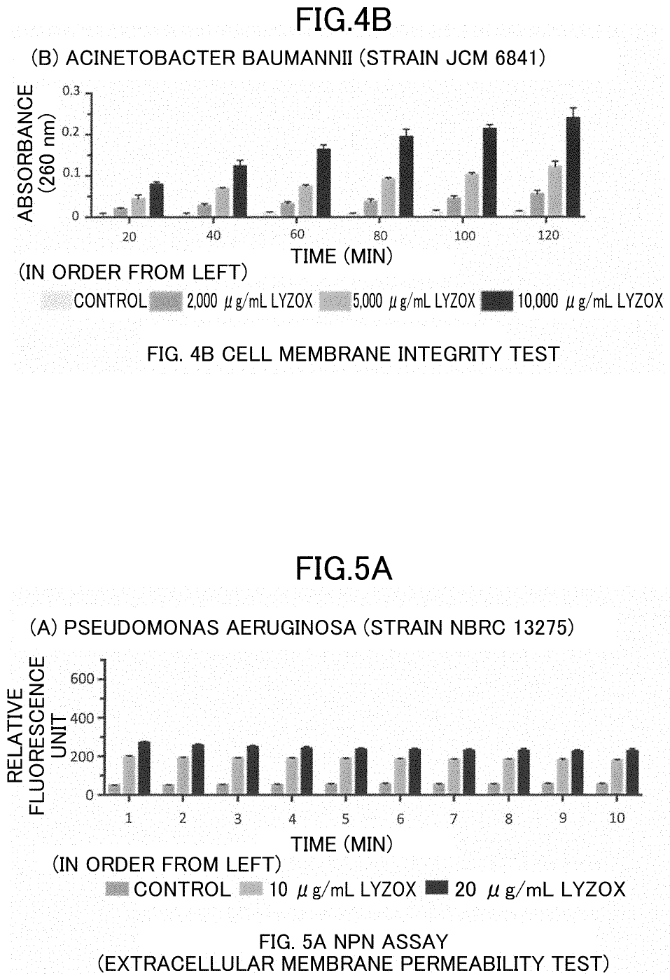

Specifically, evaluation on whether substances with absorption at 260 nm were released from the bacteria was performed by measuring the absorption at 260 nm with ND-1000 manufactured by Thermo Fisher Scientific. Solutions with different concentrations (4,000 μg/mL, 10,000 μg/mL, and 20,000 μg/mL (final concentrations after mixing with bacterial suspension were 2,000 μg/mL, 5,000 μg/mL, and 10,000 μg/mL)) were prepared as the above-described lysozyme-chitosan complex (LYZOX (registered trademark)) solution, using a 0.5% NaCl solution as the solvent, and 60 μL of 1.0 M phosphate buffered saline (pH 7.2) was further added per 1.0 mL of the solution to adjust to a neutral pH (6.6 to 7.1), thereby preparing each solution. As a control, a 0.5% NaCl solution was used. The Pseudomonas aeruginosa bacterial suspension and Acinetobacter bacterial suspension prepared in the same manner as in Experimental Example 1 were resuspended in a 0.5% NaCl solution, and prepared with such a final suspension concentration that the absorbance at 420 nm was 0.7. To 10 mL of each solution of lysozyme-chitosan complex or the like above, the above Acinetobacter bacterial suspension or Pseudomonas aeruginosa bacterial suspension was added, and incubated at 37° C. using a water bath. At 20 minutes, 40 minutes, 60 minutes, 80 minutes, 100 minutes, and 120 minutes after the start of incubation, each solution was subjected to a 0.22 μm syringe filter to remove bacteria, and the absorbance at 260 nm (A₂₆₀ₙₘ) was measured. The results for absorbance, expressed as the average of triplicate measurements and SEM (standard error of sample mean), are shown in FIGS. 4A and 4B.

The results showed that the absorbance 260 nm increased in a concentration-dependent manner (FIG. 4). When the bacterial suspensions were treated with the complex of lysozyme and chitosan bound together at various concentrations, the absorbance 260 nm increased rapidly in the first 20 minutes, and then the rate of increase decreased until 120 minutes. Thus, the lysozyme-chitosan complex showed higher absorbance in Acinetobacter compared to the control.

### Experimental Example 6 (NPN Assay Test)

Extracellular membrane permeability was determined by NPN assay (1-N-phenylnaphthylamine (NPN) uptake assay). 1-N-phenylnaphthylamine fluoresces strongly in phospholipid environments, but fluoresces weakly in aqueous environments. This property has been used to evaluate the permeability of the bacterial extracellular membrane, that is, membrane damage.

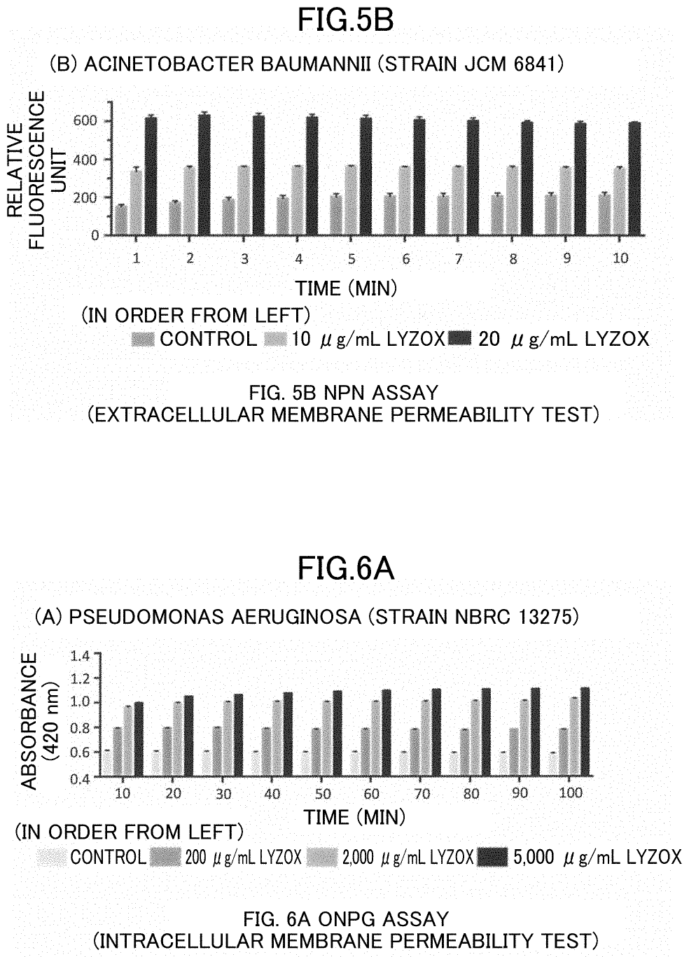

Specifically, the samples were evaluated by measuring the fluorescence intensity at an excitation wavelength of 350 nm and an emission wavelength of 420 nm using a fluorescence spectrophotometer (RF-6000 manufactured by Shimadzu Corporation). Solutions with different concentrations (20 μg/mL and 40 μg/mL (final concentrations after mixing with bacterial suspension were 10 μg/mL and 20 μg/mL)) were prepared as the above-described lysozyme-chitosan complex (LYZOX (registered trademark)) solution, using a 0.5% NaCl solution as the solvent, and 60 μL of 1.0 M phosphate buffered saline (pH 7.2) was further added per 1.0 mL of the solution to adjust to a neutral pH (7.2 to 7.3), thereby preparing each solution. As a control, a 0.5% NaCl solution was used. To these solutions, 30 μL of 1.0 mM NPN solution was added. The Pseudomonas aeruginosa bacterial suspension and Acinetobacter bacterial suspension prepared in the same manner as in Experimental Example 1 were resuspended in a 0.5% NaCl solution, and prepared with such a final suspension concentration that the absorbance at 420 nm was 1.0. To each solution of lysozyme-chitosan complex or the like above added with NPN solution, the above Acinetobacter bacterial suspension or Pseudomonas aeruginosa bacterial suspension was added, and incubated in room air, and the fluorescence intensity of each solution was measured for 10 minutes with the above fluorescence spectrophotometer at an excitation wavelength of 350 nm and an emission wavelength of 420 nm. The measurements were performed three times, and the results were obtained as relative fluorescence units (RFU) by subtracting the fluorescence intensity of the solution without bacteria from the fluorescence intensity of the solution with bacteria. The results are shown in FIGS. 5A and 5B.

In each species of bacteria, the fluorescence intensity increased after 1 minute and then remained at the same level. The lysozyme-chitosan complex showed a concentration-dependent growth suppression effect. The lysozyme-chitosan complex was shown to increase the extracellular membrane permeability within 1 minute of contact with Acinetobacter. The lysozyme-chitosan complex was found to increase the extracellular membrane permeability within 1 minute of contact with Acinetobacter. Experimental Example 7 (ONPG Assay Test)

Intracellular membrane permeability was determined by ONPG assay (o-nitrophenyl-β-D-galactopyranoside (ONPG) assay). When the cell membrane is damaged, intracellular enzymes, including β-galactosidase, leak out of the cell. ONPG is normally colorless, but is hydrolyzed by β-galactosidase to galactose and o-nitrophenol, resulting in an increase in absorbance at 420 nm. The intracellular membrane permeability of bacteria can be determined by measuring the activity of cytosolic β-galactosidase released from the bacteria using ONPG as a substrate.

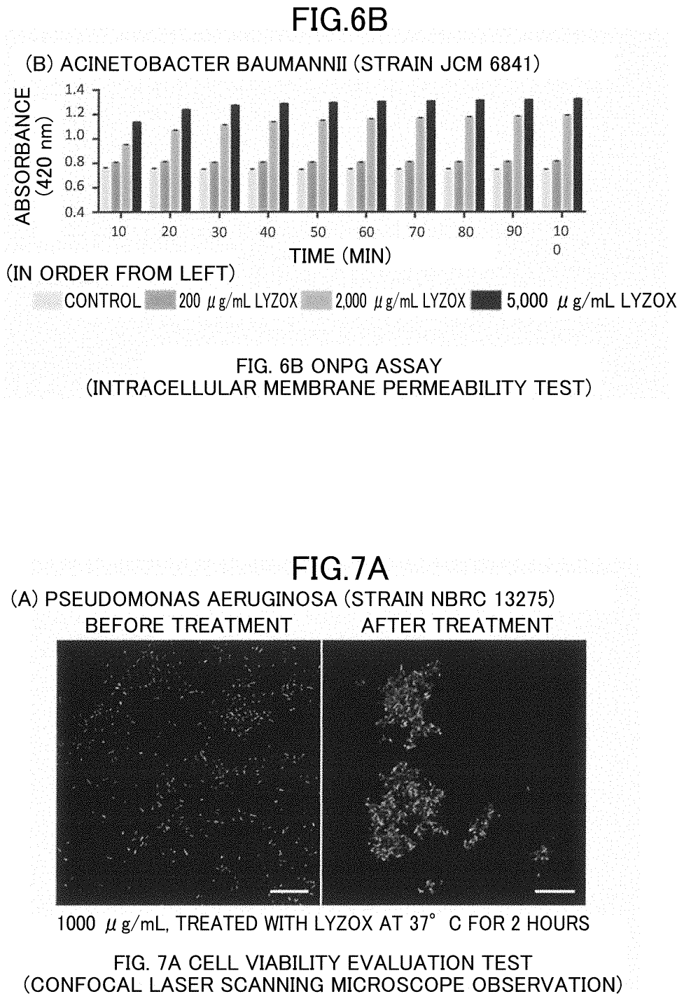

Specifically, evaluation on whether substances with absorption at 420 nm were released was performed by measuring the absorption at 420 nm with the spectrophotometer ASV11D. Solutions with different concentrations (400 μg/mL, 4,000 μg/mL, and 10,000 μg/mL (final concentrations after mixing with bacterial suspension were 200 μg/mL, 2,000 μg/mL, and 5,000 μg/mL)) were prepared as the above-described lysozyme-chitosan complex (LYZOX (registered trademark)) solution, using a 0.5% NaCl solution as the solvent, and 60 μL of 1.0 M phosphate buffered saline (pH 7.2) was further added per 1.0 mL of the solution to adjust to a neutral pH (6.8 to 7.3), thereby preparing each solution. As a control, a 0.5% NaCl solution was used. The Pseudomonas aeruginosa bacterial suspension and Acinetobacter bacterial suspension prepared in the same manner as in Experimental Example 1 were resuspended in a 0.5% NaCl solution, and prepared with such a final suspension concentration that the absorbance at 420 nm was 1.2. Each solution of lysozyme-chitosan complex or the like above in an amount of 1.6 mL, 150 μL of 30 mM ONPG solution, and 1.6 mL of the above Acinetobacter bacterial suspension or Pseudomonas aeruginosa bacterial suspension were mixed, incubated in room air, and the absorbance was measured every 10 minutes until 100 minutes after the start. The increase in absorbance at 420 nm (A₄₂₀ₙₘ) was evaluated by performing triplicate measurements. The results are shown in FIGS. 6A and 6B.

For Acinetobacter, the absorbance 420 nm in the case of using lysozyme-chitosan complex (2,000 μg/mL and 5,000 μg/mL) increased rapidly at first and then slowly until 100 minutes. In Acinetobacter treated with lysozyme-chitosan complex (200 μg/mL), the absorbance 420 nm increased in the first 10 minutes and remained constant thereafter. In each species of bacteria, the absorbance 420 nm increased in a concentration-dependent manner. The intracellular enzymes leaked out of the cells in a concentration-dependent manner within 10 minutes after contact. The lysozyme-chitosan complex was found to disrupt the inner membrane of Acinetobacter.

### Experimental Example 8 (Confocal Laser Scanning Microscope Observation)

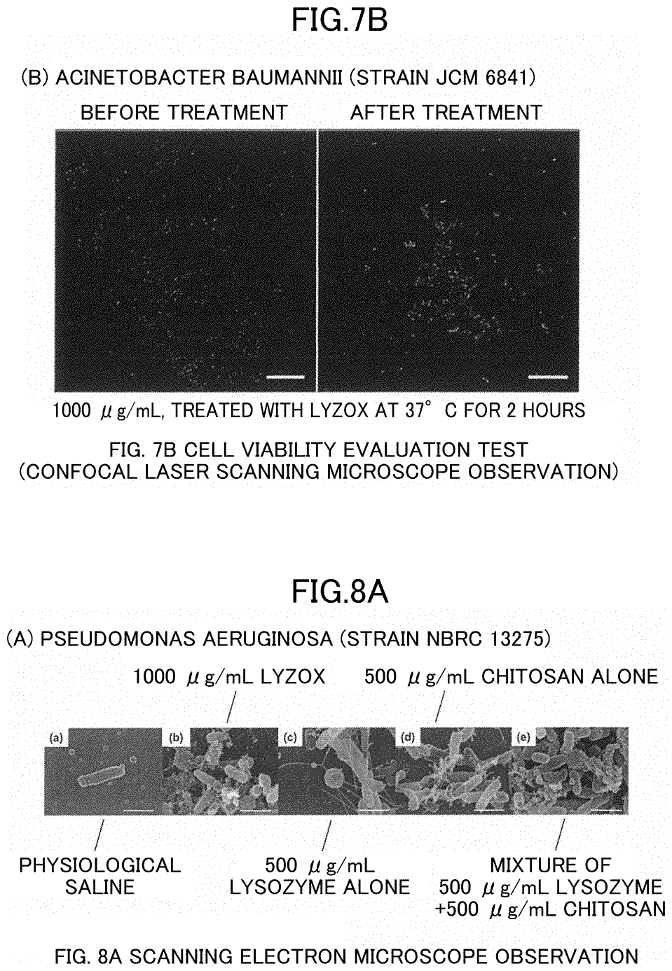

A confocal laser scanning microscope (CLSM) was used to visualize the damage to the bacterial cell membrane after treatment with the lysozyme-chitosan complex (scale bar in FIG. 7=25 μm).

Specifically, a 2,000 μg/mL solution (final concentration after mixing with bacterial suspension was 1,000 μg/mL) of the lysozyme-chitosan complex described above (LYZOX (registered trademark)) was prepared using physiological saline (0.9% NaCl solution) as a solvent, and 60 μL of 1.0 M phosphate buffered saline (pH 7.2) was further added per 1.0 mL of the solution to adjust the pH to neutral (pH 7.0 to 7.1), thereby preparing the lysozyme-chitosan complex solution. As a control, physiological saline (0.9% NaCl solution) was used. To 500 μL of these solutions, 500 μL of Pseudomonas aeruginosa bacterial suspension and Acinetobacter bacterial suspension prepared in the same manner as in Experimental Example 1 above was added, and incubated at 37° C. for 2 hours. Then, 3 μL of LIVE/DEAD BacLight reagent (a mixture of SYTO 9 and propidium iodide (PI), which stain nucleic acids) was added and further incubated for 15 minutes at room temperature. SYTO 9 with green fluorescence labels all bacteria, while PI with red fluorescence permeates only bacteria with damaged plasma membranes, and the presence of both dyes attenuates the fluorescence of SYTO 9. The excitation wavelength/emission wavelength were observed at 488/495 to 515 nm for SYTO 9 and 488/635 to 700 nm for PI (FIGS. 7A and 7B).

In the control, almost all bacteria were intact (green fluorescence), but a few had damaged membranes (red fluorescence). In each sample treated with the lysozyme-chitosan complex, there were more bacterial cells with damaged membranes than in the corresponding control (red fluorescence predominated). In Pseudomonas aeruginosa treated with the lysozyme-chitosan complex, intact bacterial cells and bacterial cells with damaged membranes aggregated to form clusters. In contrast, Acinetobacter treated with the lysozyme-chitosan complex showed less aggregation of bacterial cells than Pseudomonas aeruginosa. The findings of CLSM were consistent with the results of previous membrane damage assays.

### Experimental Example 9 (Scanning Electron Microscope Observation)

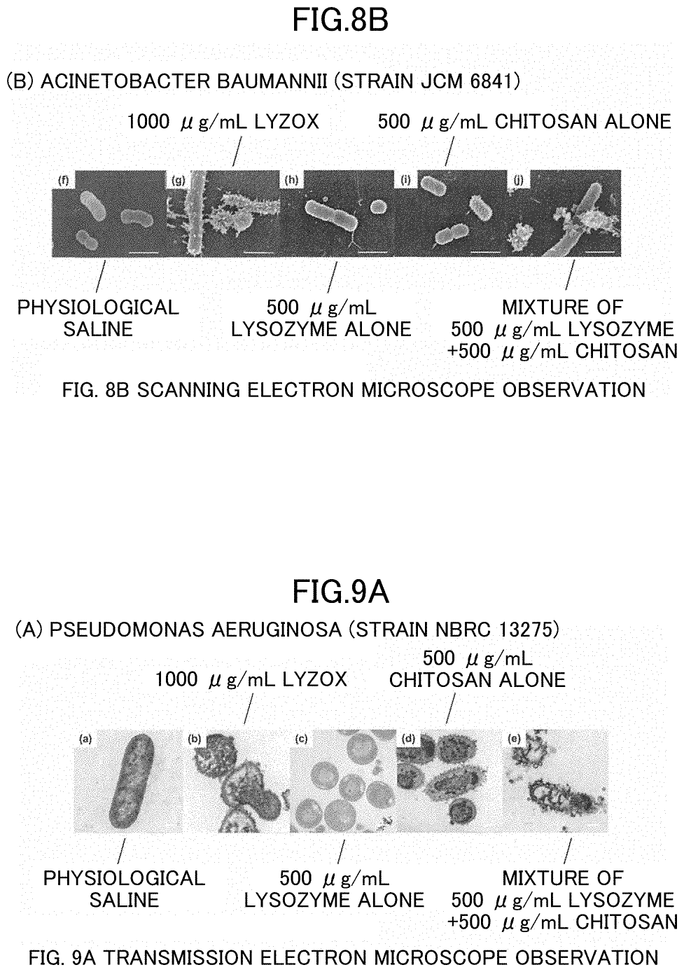

The morphological changes of Pseudomonas aeruginosa and Acinetobacter by the lysozyme-chitosan complex were observed with a scanning electron microscope (SEM) (photographed at 20000-time magnification, scale bar=1.5 μm in FIG. 8).

Specifically, the above-mentioned lysozyme-chitosan complex (LYZOX (registered trademark)) solution (1,000 μg/mL), a solution of lysozyme alone (500 μg/mL), a solution of chitosan alone (500 μg/mL), and a solution of a mixture of lysozyme and chitosan (solution of lysozyme alone [500 μg/mL] and solution of chitosan alone [500 μg/mL]) were prepared using physiological saline (0.9% NaCl solution) as a solvent. As a control, physiological saline (0.9% NaCl solution) was used. To the Pseudomonas aeruginosa bacterial suspension and Acinetobacter bacterial suspension prepared in the same manner as in Experimental Example 1 above except that the absorbance was set at 2.0, with all the supernatant removed, 1 mL of each solution of lysozyme-chitosan complex or the like above was added and incubated at 37° C. for 2 hours. Then, centrifugation was performed, and the resultant pellets were washed with physiological saline (0.9% NaCl) and fixed in a fixative solution (2.5% buffered glutaraldehyde in a 0.1 M phosphate buffered saline) at 4° C. for 2 hours. Then, they were incubated in a 0.1 M phosphate buffered saline overnight.

The bacteria were then fixed in 1.0% buffered osmium tetroxide in a 0.1 M phosphate buffered saline for 1 hour, followed by dehydration with ethanol and drying using a critical point drying device (HCP-2). After coating with about 20 nm of platinum/palladium, the samples were observed with a scanning electron microscope (S-4500) (FIGS. 8A and 8B).

The SEM findings of untreated Pseudomonas aeruginosa (NBRC 13275) (FIG. 8A) and Acinetobacter baumannii (FIG. 8B) showed smooth surfaces ((a) of FIG. 8A and (f) of FIG. 8B). After treatment with lysozyme alone, Pseudomonas aeruginosa and Acinetobacter formed spheroplasts, which are partially spherical cells ((c) of FIG. 8A and (h) of FIG. 8B). In both Pseudomonas aeruginosa and Acinetobacter treated with lysozyme-chitosan complex ((b) of FIG. 8A and (g) of FIG. 8B), more vesicles were observed compared to those treated with chitosan alone and a mixture ((d) and (e) of FIG. 8A, and (i) and (j) of FIG. 8B). Pseudomonas aeruginosa and Acinetobacter treated with lysozyme-chitosan complex and mixture lost the integrity of their surface structures and became partially spherical ((b) and (e) of FIG. 8A, and (g) and (j) of FIG. 8B). After treatment with each solution, especially with lysozyme-chitosan complex, extracellular filamentous structures appeared around the cells ((b) to (e) of FIG. 8A, and (g) to (j) of FIG. 8B).

### Experimental Example 10 (Transmission Electron Microscope Observation)

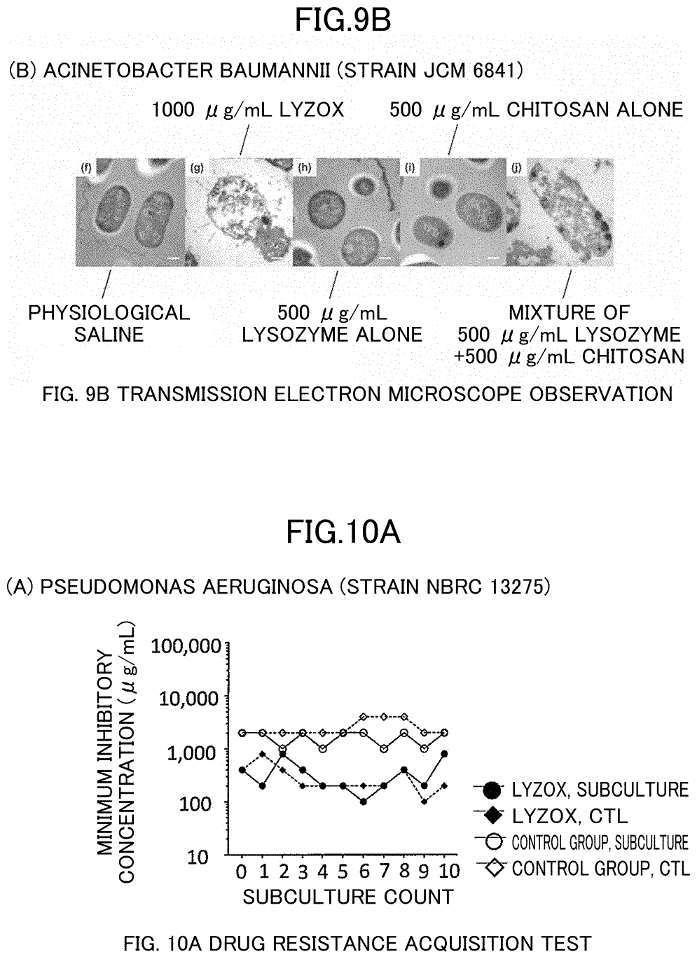

The morphological changes of Pseudomonas aeruginosa and Acinetobacter by the lysozyme-chitosan complex were observed with a transmission electron microscope (TEM) (photographed at 50,000-time magnification, scale bar=0.2 μm in FIG. 9).

Specifically, the above-mentioned lysozyme-chitosan complex (LYZOX (registered trademark)) solution (1,000 μg/mL), a solution of lysozyme alone (500 μg/mL), a solution of chitosan alone (500 μg/mL), and a solution of a mixture of lysozyme and chitosan (solution of lysozyme alone [500 μg/mL] and solution of chitosan alone [500 μg/mL]) were prepared using physiological saline (0.9% NaCl solution) as a solvent. As a control, physiological saline (0.9% NaCl solution) was used.

To 1 mL of these solutions, the Pseudomonas aeruginosa bacterial suspension and Acinetobacter bacterial suspension prepared in the same manner as in Experimental Example 1 above were diluted with physiological saline (0.9% NaCl) so that the final suspension concentration was an absorbance of 4.0 at 600 nm, and were prepared by removing all the supernatant, and to these, 1 mL of each solution of the lysozyme-chitosan complex or the like above was added and incubated at 37° C. for 2 hours. Then, centrifugation was performed, and the resultant pellets were washed with physiological saline (0.9% NaCl), fixed in a 1.0% buffered osmium tetroxide buffer solution in a 0.1 M phosphate buffered saline for 1 hour, embedded in 2.0% agar, dehydrated with ethanol, and then embedded in Epon 812 (TAAB Laboratories Equipment Ltd.). Ultrathin sections of 70 nm were collected on a copper grid, double-stained with uranyl acetate and lead citrate, and observed with a transmission electron microscope (H-7100) at 75 kV.

TEM findings of untreated Pseudomonas aeruginosa and Acinetobacter show normal bacterial surface structures, which appear smooth and layered ((a) of FIG. 9A, and (f) of FIG. 9B). When treated with lysozyme alone, both Pseudomonas aeruginosa and Acinetobacter formed spheroplasts ((c) of FIG. 9A, and (h) of FIG. 9B), and when treated with chitosan alone, these bacteria formed numerous vesicles on their surfaces ((d) of FIG. 9A, and (i) of FIG. 9B). The vesicles were generated from the outer membranes, and these were consistent with the vesicles observed in the SEM of the bacteria. After treatment with the lysozyme-chitosan complex or a mixture, the TEM findings of Pseudomonas aeruginosa and Acinetobacter baumannii revealed morphological changes resulting from the treatment with both lysozyme alone and chitosan alone by showing spherical cells with outer membrane vesicles ((b) and (e) of FIG. 9A, and (g) and (j) of FIG. 9B). Furthermore, the bacterial cell walls were disrupted and the intracellular contents were released from the cells.

### Experimental Example 11 (Drug Resistance Acquisition Test)

To evaluate the acquisition of drug resistance, Pseudomonas aeruginosa (NBRC 13275 and PAO 1) and Acinetobacter baumannii were repeatedly subcultured in LB medium containing lysozyme-chitosan complex, and the changes in sensitivity to lysozyme-chitosan complex were evaluated.

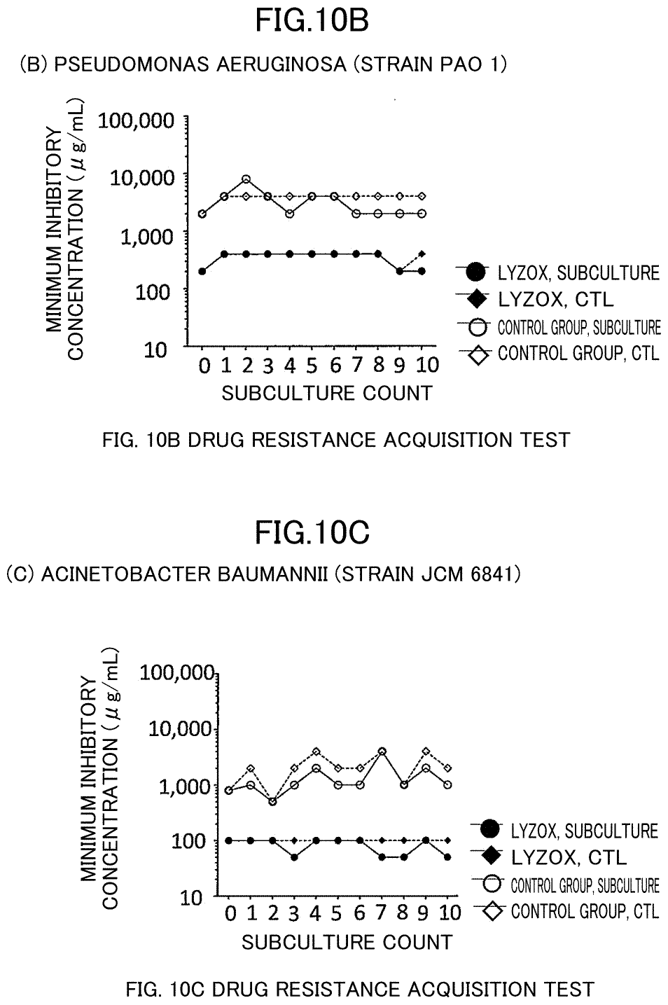

Specifically, for the MIC (minimum inhibitory concentration: the minimum amount of drug required to inhibit bacterial growth) of the lysozyme-chitosan complex, Pseudomonas aeruginosa (NBRC 13275 and PAO 1) or Acinetobacter baumannii was adjusted using physiological saline (NaCl 0.9%) as a solvent such that the absorbance was 1.0 at 600 nm, and 60 μL of the adjusted bacterial mixture was added to 140 μL of LB medium in a 96-well microplate to prepare a final bacterial concentration of 10⁴ in one well, which was incubated at 35° C. The MIC was defined as the minimum concentration of the reagent without visual turbidity observed after 18 hours and 24 hours of evaluation. Bacteria were repeatedly subcultured (10 times) at a lysozyme-chitosan complex concentration of ½ MIC, which is half of the MIC, and evaluated for each MIC (LYZOX in FIGS. 10A and 10B, subculture). As a comparison, the above mixture of lysozyme and chitosan was used (control groups in FIGS. 10A and 10B), and the same tests as for the lysozyme-chitosan complex were performed (control groups in FIGS. 10A and 10B, subculture). The bacteria that were not subcultured was evaluated as a control (CTL in FIGS. 10A and 10B). MIC measurements were repeated 10 times to evaluate the development of drug resistance in these bacteria.

The obtained drug resistance tests are shown in FIG. 10. After 10 subcultures, Pseudomonas aeruginosa and Acinetobacter did not acquire resistance to the lysozyme-chitosan complex and its mixture.

### Experimental Example 12 (Hemolytic Toxicity Test)

To confirm the safety of the lysozyme-chitosan complex, a hemolytic toxicity test was performed.

Specifically, 400 μL of rabbit defibrotic blood (obtained from Cosmo Bio Co., Ltd.) was diluted using phosphate buffered saline (PBS) as a solvent to prepare 20 μg/mL, 200 μg/mL, 2,000 μg/mL, and 20,000 μg/mL (as shown in Table 1, the concentrations during the hemolytic toxicity evaluation test were 10 μg/mL, 100 μg/mL, 1,000 μg/mL, and 10,000 μg/mL (Table 1)) lysozyme-chitosan complex (LYZOX (registered trademark)) solutions. An additional 500 μL of the above rabbit defibrotic blood diluted in PBS above was prepared and added to 500 μL of each lysozyme-chitosan complex (LYZOX (registered trademark)) solution, PBS (negative control), and 0.1% Triton-X 100 solution (positive control). Each resulting mixed solution was incubated at 37° C. for 1 hour, and centrifuged at 1500 rpm for 10 minutes, and the supernatant was collected. Each supernatant in an amount of 750 μL was added to 750 μL of PBS above, and 750 μL of the control (negative control or positive control) was added to 750 μL of each corresponding concentration of lysozyme-chitosan complex. Each absorbance (OD) was measured at 545 nm to calculate the hemolytic rate by using the following formula. The absorbance value at 545 nm was expressed as the average and SEM (standard error of the sample mean) of triplicate measurements. Hemolytic rate (HR) (%)=(absorbance of sample [AS]-negative control absorbance of corresponding concentration [AN])/(positive control absorbance corresponding to lysozyme-chitosan complex concentration [AP]-[AN])×100.

TABLE 1
Hemolytic Toxicity Test

| | Absorbance of | | | |
|---|---|---|---|---|
| | Lysozyme- | | | |
| Lysozyme- | Chitosan | Positive | Negative | |
| Chitosan | Complex | Control | Control | Hemolytic |
| Complex | Sample | Absorbance | Absorbance | Rate |
| Concentration | [AS] | [AP] | [AN] | (%) |
| 10,000 μg/mL | 0.489 ± 0.005 | 1.458 ± 0.022 | 0.457 ± 0.012 | 2.17 ± 0.64 |
| 1,000 μg/mL | 0.155 ± 0.008 | 1.161 ± 0.013 | 0.130 ± 0.006 | 2.19 ± 0.26 |
| 100 μg/mL | 0.097 ± 0.004 | 1.091 ± 0.010 | 0.086 ± 0.007 | 1.05 ± 0.70 |
| 10 μg/mL | 0.090 ± 0.010 | 1.027 ± 0.020 | 0.084 ± 0.009 | 0.55 ± 0.26 |

As shown in Table 1, for the lysozyme-chitosan complex, rabbit erythrocytes almost did not hemolyze after 1 hour of incubation at 37° C. The hemolytic rate of the lysozyme-chitosan complex was less than 5% at concentrations in the range of 10 μg/mL to 10,000 μg/mL. Note that it has been reported that a hemolytic rate of 5% or less is acceptable for clinical biomaterials and is safe [Deng J D, He B, He D H, Chen Z F. A potential biopreservative: Chemical composition, antibacterial and hemolytic activities of leaves essential oil from Alpinia guinanensis. Ind Crop Prod. 2016; 94:281-7.].

### INDUSTRIAL APPLICABILITY

According to the present invention, it is possible to provide a novel antibacterial composition, pharmaceutical composition, and the like against Acinetobacter.

1. An antibacterial composition comprising: a complex of lysozyme and chitosan bound together, with antibacterial property against Acinetobacter.

2. The antibacterial composition according to claim 1, wherein the Acinetobacter is multidrug-resistant Acinetobacter.

3. The antibacterial composition according to claim 1, wherein a concentration of the complex in 1 mL of the antibacterial composition is 200 μg/mL or more.

4. The antibacterial composition according to claim 1, wherein the antibacterial property has bacteria-killing property.

5. Rinse water comprising: the antibacterial composition according to claim 1.

6. A pharmaceutical composition for treating or preventing an Acinetobacter infection, comprising: a complex of lysozyme and chitosan bound together.

7. The pharmaceutical composition according to claim 6, wherein the Acinetobacter infection is a multidrug-resistant Acinetobacter infection.

8. Use of a complex of lysozyme and chitosan bound together in production of a pharmaceutical composition for treating or preventing an Acinetobacter infection.

9. A method for treating or preventing an Acinetobacter infection, comprising a step of administering to a subject a pharmaceutical composition containing a complex of lysozyme and chitosan bound together.

10. The method according to claim 9, wherein the Acinetobacter is multidrug-resistant Acinetobacter.

11. The method according to claim 9, wherein a concentration of the complex in 1 mL of the antibacterial composition is 200 μg/mL or more.

12. The method according to claim 9, wherein the chitosan is a water-soluble chitosan having a molecular weight of 1000 Da to 30,000 Da.

13. The method according to claim 9, wherein the complex is contained at 0.01 mg/ml to 50 mg/ml in the pharmaceutical composition.

14. The method according to claim 9, wherein the complex is contained at 0.001% by mass to 5.0% by mass in the pharmaceutical composition.

15. The method according to claim 9, wherein the pharmaceutical composition is administered at the daily dose of 0.1 to 20 mg/kg body weight.
